(19) United States
(12) Patent Application Publication
(10) Pub. No.: US 2025/0166398 A1
(43) Pub. Date: May 22, 2025

(54) ARTIFICIAL INTELLIGENCE FOR IMAGING FLOW CYTOMETRY

(71) Applicant: CYTEK BIOSCIENCES, INC., Fremont, CA (US)

(72) Inventors: Vidya Venkatachalam, Fremont, CA (US); Paula Glaus, Fremont, CA (US); Alan Yang Li, Fremont, CA (US)

(73) Assignee: CYTEK BIOSCIENCES, INC., Fremont, CA (US)

(21) Appl. No.: 18/749,606

(22) Filed: Jun. 20, 2024

Related U.S. Application Data
(60) Provisional application No. 63/522,133, filed on Jun. 20, 2023.
(60) Provisional application No. 63/522,398, filed on Jun. 21, 2023.
(60) Provisional application No. 63/522,400, filed on Jun. 21, 2023.

(51) Int. Cl. G06V 20/69; G06V 10/40; G06V 10/764; G06V 10/82; G16B 15/10

(52) U.S. Cl. CPC G06V 20/698; G06V 10/40; G06V 10/764; G06V 10/82; G06V 20/693; G06V 20/695; G16B 15/10

(57) ABSTRACT

A multispectral imaging flow cytometer acquires a variety of images in different imaging modes, such as brightfield, side scatter, and a plurality of fluorescent images of a different moving biological cells in a sample fluid. These images can be processed by a plurality of artificial intelligence algorithms and/or machine learning tools executed by a processor, a neural engine, a neural processor, or a convolutional neural network (CNN). Deep learning analysis of the images can be performed with the CNN on the images to extract image features. Feature data can be extracted about the moving biological cell as well. An AI algorithm, such as random forest algorithm, can use both the image features of a cell and the feature data of the cell to classify the biological cell as to its type.

A portion of the disclosure of this patent document contains material to which a claim for copyright and trademark is made. The copyright and trademark owner has no objection to the reproduction of the patent document or the patent disclosure, as it appears in the U.S. Patent Office records, but reserves all other copyright and trademark rights whatsoever.

### CROSS-REFERENCE TO RELATED APPLICATIONS

This patent application claims the benefit of U.S. Provisional Patent Application No. 63/522,133 titled “ARTIFICIAL INTELLIGENCE FOR IMAGING FLOW CYTOMETRY” filed on Jun. 20, 2023 by inventor Vidya Venkatachalam et al., incorporated herein for all intents and purposes. This patent application claims the benefit of U.S. Provisional Patent Application No. 63/522,398 titled “METHODS OF ARTIFICIAL INTELLIGENCE FOR IMAGING FLOW CYTOMETRY” filed on Jun. 21, 2023 by inventor Vidya Venkatachalam et al., incorporated herein for all intents and purposes. This patent application claims the benefit of U.S. Provisional Patent Application No. 63/522,400 titled “SYSTEMS FOR ARTIFICIAL INTELLIGENCE FOR IMAGING FLOW CYTOMETRY” filed on Jun. 21, 2023 by inventor Vidya Venkatachalam et al., incorporated herein for all intents and purposes.

This patent application is further a continuation in part and claims the benefit of U.S. patent application Ser. No. 18/647,366 titled COMBINING BRIGHTFIELD AND FLUORESCENT CHANNELS FOR CELL IMAGE SEGMENTATION AND MORPHOLOGICAL ANALYSIS IN IMAGES OBTAINED FROM AN IMAGING FLOW CYTOMETER filed by inventors Alan Li et al on Apr. 26, 2024, incorporated herein for all intents and purposes. U.S. patent application Ser. No. 18/647,366 is a continuation of U.S. patent application Ser. No. 17/076,008 titled METHOD TO COMBINE BRIGHTFIELD AND FLUORESCENT CHANNELS FOR CELL IMAGE SEGMENTATION AND MORPHOLOGICAL ANALYSIS USING IMAGES OBTAINED FROM IMAGING FLOW CYTOMETER (IFC) filed by inventors Alan Li et al on Dec. 16, 2022, incorporated herein for all intents and purposes.

This application incorporated by reference U.S. patent application Ser. No. 17/016,244 titled USING MACHINE LEARNING ALGORITHMS TO PREPARE TRAINING DATASETS filed on Sep. 9, 2020 by inventors Bryan Richard Davidson et al. for all intents and purposes. For all intents and purposes, Applicant incorporates by reference in their entirety the following U.S. Pat. Nos. 6,211,955, 6,249,341, 6,256,096, 6,473,176, 6,507,391, 6,532,061, 6,563,583, 6,580,504, 6,583,865, 6,608,680, 6,608,682, 6,618,140, 6,671,044, 6,707,551, 6,763,149, 6,778,263, 6,875,973, 6,906,792, 6,934,408, 6,947,128, 6,947,136, 6,975,400, 7,006,710, 7,009,651, 7,057,732, 7,079,708, 7,087,877, 7,190,832, 7,221,457, 7,286,719, 7,315,357, 7,450,229, 7,522,758, 7,567,695, 7,610,942, 7,634,125, 7,634,126, 7,719,598, 7,889,263, 7,925,069, 8,005,314, 8,009,189, 8,103,080, and 8,131,053.

### FIELD

The embodiments of the invention relate generally to artificial intelligence to detect and classify images of biological cells flowing in a fluid captured by an imaging flow cytometer.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1A is a block diagram of an embodiment of an artificial intelligence (AI) imaging flow cytometer system.

FIG. 1B is a schematic view of a computer network for deploying embodiments.

FIG. 1C is a block diagram of a computer node in the computer network environment shown in FIG. 1B.

FIGS. 2A-2B illustrate the two types of data inputs into the AI imaging analysis device/software.

FIG. 3A illustrate using a brightfield image and a fluorescent image of four different cell images.

FIG. 3B illustrates a table of example numeric features broken up by category.

FIG. 4 illustrates a first AI algorithm used to process each of the multispectral cellular images with a convolutional neural network (CNN) for image processing and image feature extraction.

FIG. 5 illustrates a random forests algorithm to gain insight into which features have the most impact on the data in an image flow cytometry experiment.

FIG. 6 illustrates a flow chart of a method of AI analysis of images captured by an imaging flow cytometer with multiple machine learning and AI algorithms.

FIG. 7A illustrates cells with micronuclei having DNA damage.

FIG. 7B illustrates cells with micronuclei having proliferation (Cytotoxicity).

FIG. 7C illustrates a set of truth series of images.

### DETAILED DESCRIPTION

In the following detailed description of the disclosed embodiments, numerous specific details are set forth in order to provide a thorough understanding. However, it will be obvious to one skilled in the art that the disclosed embodiments may be practiced without these specific details. In other instances, well known methods, procedures, components, and subsystems have not been described in detail so as not to unnecessarily obscure aspects of the disclosed embodiments.

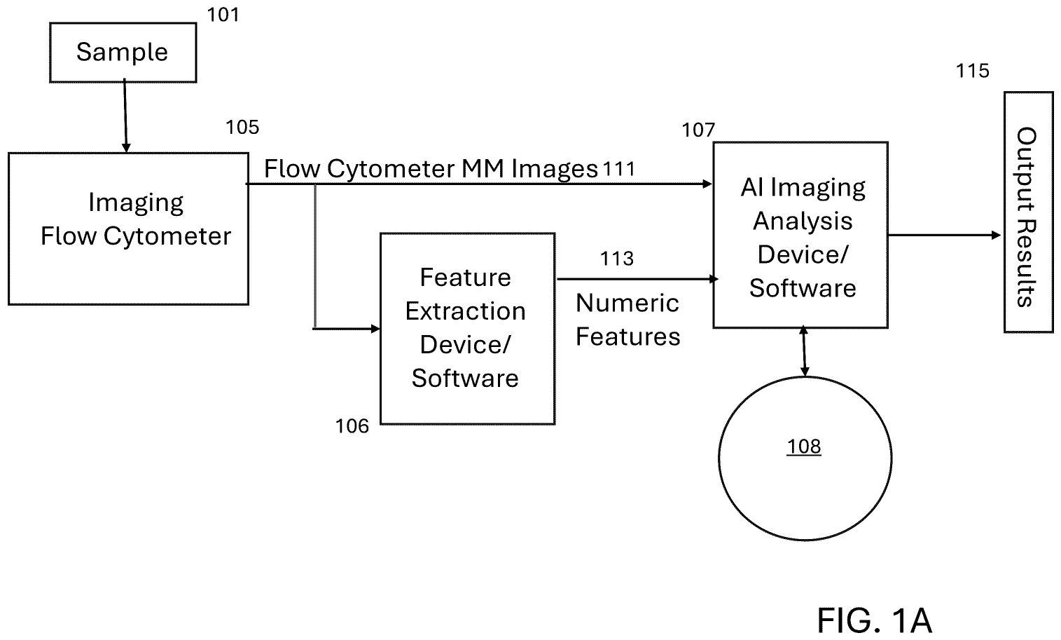

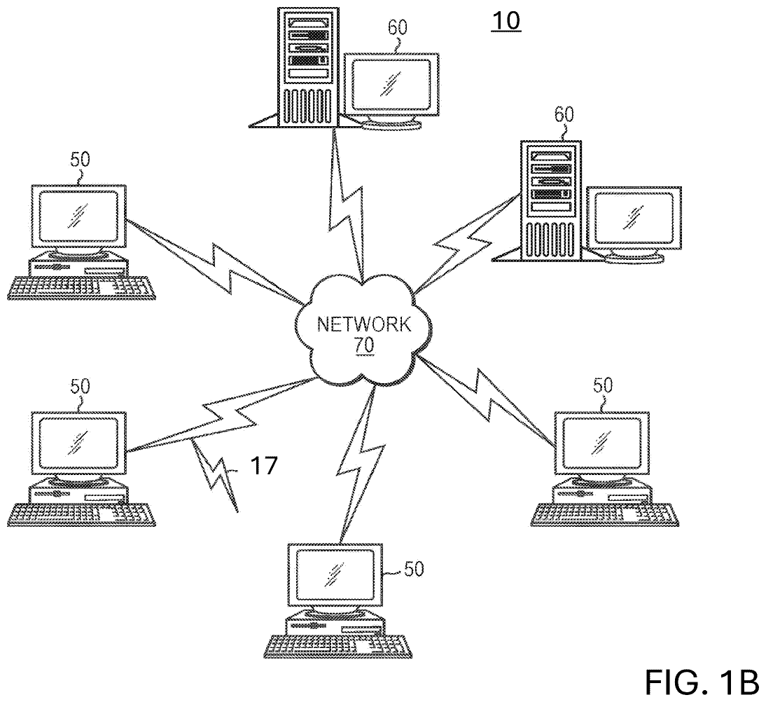

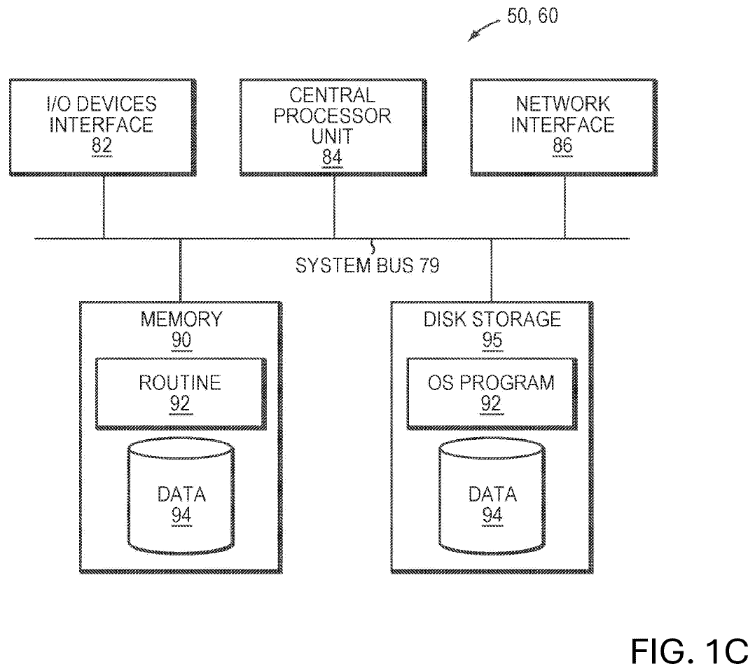

FIG. 1 illustrates an artificial intelligence (AI) imaging flow cytometer system 100. The system 100 includes a multispectral imaging flow cytometer 105, a feature extraction device/software process 106, and an AI imaging analysis device/software process 107. The feature extraction device (extractor) 106 and the AI imaging analysis device (analyzer) 107 are both computer implemented devices formed by the execution of software. The AI imaging analysis device (analyzer) 107 is coupled in communication with a storage device 108 to store files of numeric feature data and associated image data, AI models, and training images and data in databases. Deep learning AI models for cell images are often trained with large cell image databases.

A biological sample 101 of interest, such as bodily fluids or other material (medium) carrying subject cells is provided as input int the multispectral imaging flow cytometer 105. The imaging flow cytometer 105 combines the fluorescence sensitivity of standard flow cytometry with the spatial resolution and quantitative morphology of digital microscopy. An example imaging flow cytometer is the AMNIS IMAGESTREAM manufactured by Applicant. Other imaging flow cytometers that can generate multi-model or multispectral images of each biological cell are suitable.

The imaging flow cytometer 105 is compatible with a broad range of cell staining protocols of conventional flow cytometry as well as with protocols for imaging cells on slides. See U.S. Pat. Nos. 6,211,955; 6,249,341; 7,522,758 and “Cellular Image Analysis and Imaging by Flow Cytometry” by David A. Basiji, et al. published in Clinical Laboratory Medicine 2007 September, Volume 27, Issue 3, pages 653-670 (herein incorporated by reference in their entirety).

The imaging flow cytometer 105 electronically tracks moving cells in the sample with a high resolution multispectral imaging system and simultaneously acquires multiple images of each target cell in different imaging modes. In one embodiment, the acquired images 121 of a cell include: a side-scatter (darkfield) image, a transmitted light (brightfield) image, and a plurality of fluorescence images of different spectral bands. Importantly, not only are the cellular images (i.e., images of a cell) simultaneously acquired but they are also spatially well aligned with each other across the different imaging modes. Thus, the acquired darkfield image, brightfield image and fluorescence images (collectively multispectral images 111) of a subject cell are spatially well aligned with each other enabling mapping of corresponding image locations to within about 1-2 pixels accuracy.

The acquired cellular multispectral images 111 are output from imaging flow cytometer 105 and coupled into the computer-implemented feature extraction device 106 and the computer-implemented AI imaging analysis device 107. For a non-limiting example, embodiments may employ an input assembly for implementing streaming feed or other access to the acquired images 111. The computer-implemented feature extraction device 106 and the computer-implemented AI imaging analysis device 107 can be configured to automatically analyze thousands of cellular images 111 in near real time of image acquisition or access, and to accurately identify different cellular and subcellular components of the sample cells being analyzed. Each multispectral image 111 of a cell has different cellular components and different subcellular components representing cells in the sample. A given cellular component may be formed of one or more image subcomponents representing parts (portions) of a cell.

The acquired cellular multispectral images 111 are coupled into both the feature extraction device 106 and the AI imaging analysis device 107. Numeric features of each cell in each multispectral image 111 are extracted by the feature extraction device 106. Image features of each cell in each multispectral image 111 are extracted by the AI imaging analysis device 107. Advanced shape image features such as contour curvature and bending scope can be determined from a brightfield image in each multispectral image 111. With both numeric features and image features, the AI imaging analysis device 107 can further classify the cell type and cell morphology of each cell in each multispectral image 111. Complex cell morphologies can be determined such as fragmented or detached cells, stretched or pointed cell boundary, etc. in the sample cells.

Based on the numerical feature inputs 113 and the acquired cellular multispectral images 111, the output results 115 of the AI imaging analysis device 107 (and thus system 100) provides indications of identified cell morphologies and/or classification of cell type. A computer display monitor or other output device (e.g., printer/plotter) may be used to render these output results 115 to an end-user.

### Why Use Machine Learning

So why use machine learning. Well, if you're me and you're a machine learning engineer the answer is of course you use machine learning. But more importantly for you we all know that manual analysis has multiple pain points. It has a very steep learning curve. You really need to know your data and you really really need to know your tools in order to create a very effective analysis that's useful to you. Additionally, you know manual analysis is very prone to bias and subjectivity. Looking at cells, everyone has their own way of doing things and sometimes you end up with differences in opinion. This really ties into the third bullet point which is there's a lack of repeatability and standardized workflows. It's very very difficult for a large number of people to all follow the same set of steps and come out with the same output. AMNIS AI offers multiple benefits to counteract some of these pain points. It has an intuitive design that's easy and effective to follow, it offers objective and repeatable analysis, it has scalable workflow options, and it's shareable across multiple users. Most importantly, AMNIS AI supports diverse data sets. From animal fertility, phytoplankton, micronuclei, you can load any data into amnesia and get started with your analysis. The most important takeaway though is AMNIS AI requires no coding knowledge. You don't need to know any type of programming in order to use AMNIS AI successfully.

### Image Flow Cytometry Analysis

Before we start talking about 2.0 let's revisit AMNIS AI 1.2 especially for those who are maybe less familiar with the software. The most important thing is that AMNIS AI uses AI-powered analysis to significantly simplify the workflow. There are a couple key features. AMNIS AI has a deep neural network model for image classification, the database is optimized to handle large data sets, you can classify your data using a pre-existing model, or you can train a new model using date your new data. In addition, of course training a model requires tagged truth data. There is an AI assisted tagging module to assist you in that process. There's an interactive results gallery so you can explore the output of your model and there's report generation to summarize it all very neatly for you. AMNIS AI builds on the intuitive and robust workflow of 1.2 it takes everything that was really excellent about that previous version and makes it even better and more effective.

So, let's introduce AMNIS AI 2.0. Now I love my what why and hows. The what is that AMNIS AI is a powerful intuitive software that allows users to build robust machine learning pipelines. It gives you access to multiple algorithms and can ingest data from the AMNIS ImageStream Mk 2 and the AMNIS FlowSight imaging Flow Cytometer. The why is that we see a need to simplify the analysis workflow, reduce ramp-up time for new users and to improve overall efficiency. We want to put machine learning in the hands of any user regardless of their technical background and how we're going to accomplish this is by providing an easy-to-follow step-by-step process that lets users efficiently tag data, utilize pre-optimized machine learning algorithms and view their concise results.

Data Inputs into AI Imaging Analysis Software

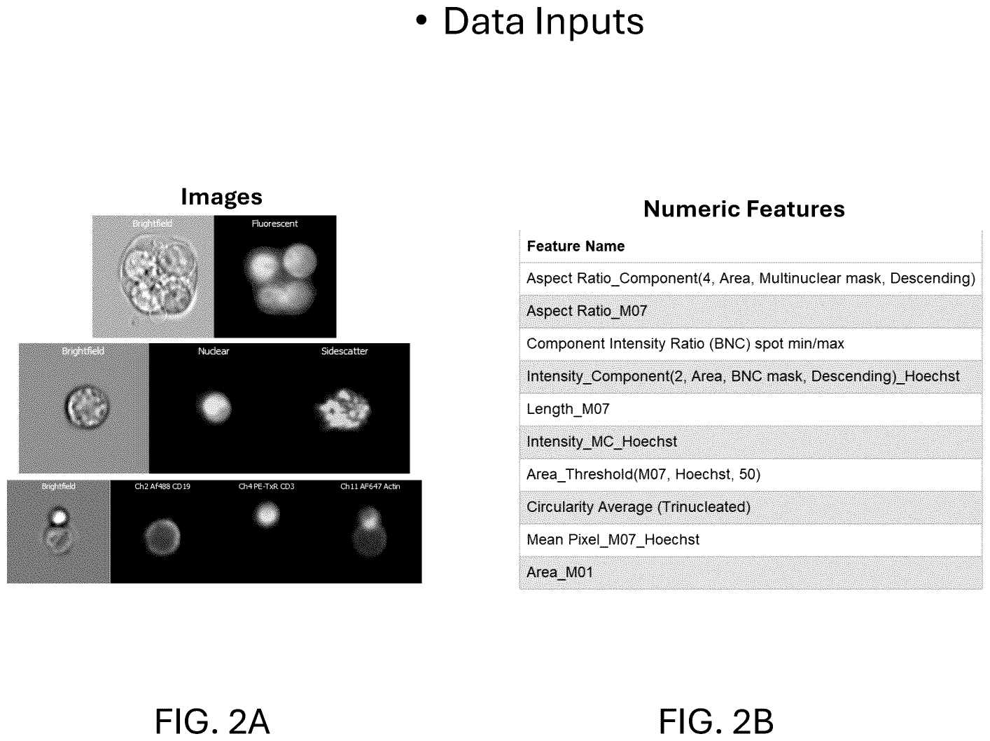

Referring now to FIGS. 2A-2B, the AI image analysis software can have two different kinds of data inputs. A first data input are images and a second data input are numeric features. Previously, only image inputs were supported. While images are effective to work with alone, powerful extracted numeric features generated by feature extraction software software can be fused together with the image feature extraction to provide more rapid and improved classification results by the AI image analysis device/software. Both images and features are supported data inputs into the AI image analysis software.

### Images

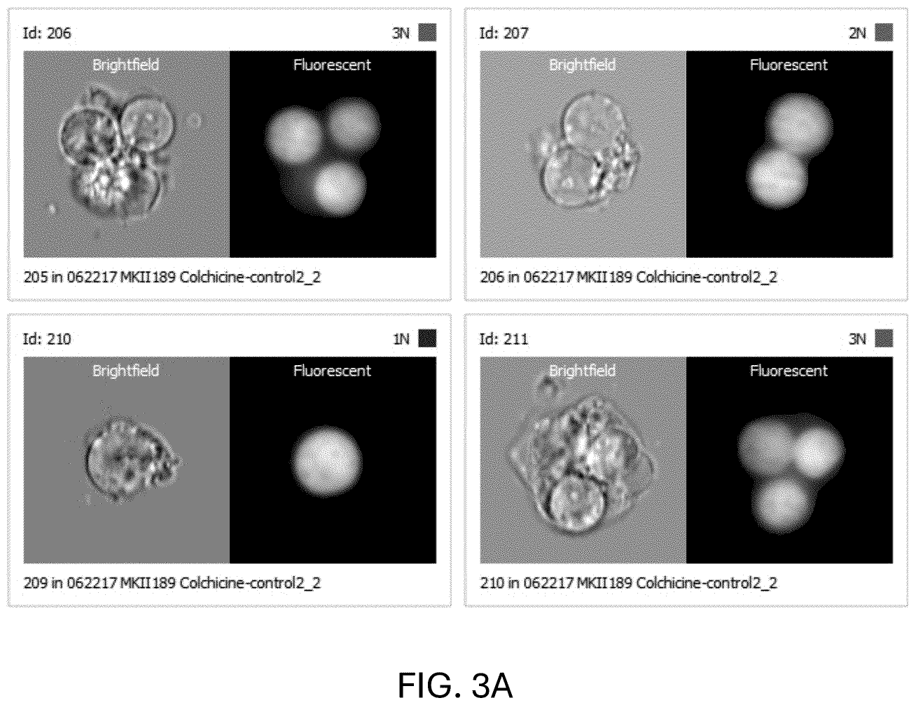

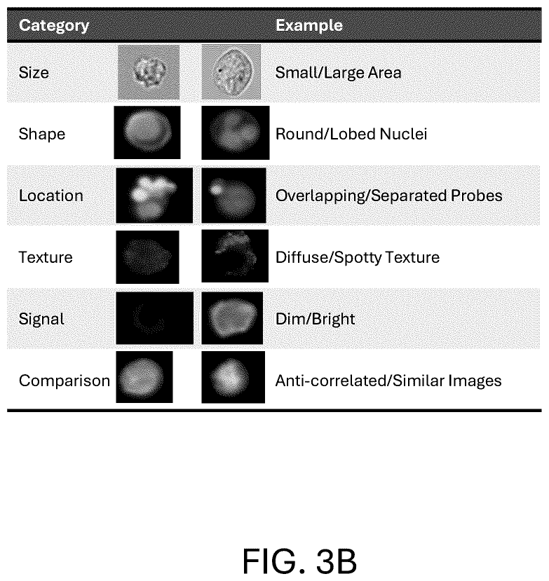

Referring now to FIG. 2A, one type of data input to the AI analysis software are images captured by the imaging flow cytometer. Each image of each cell are multispectral images captured using different modalities. These can be a brightfield image, darkfield image, nuclear image (cell nucleus), side scatter image, and one or more fluorescent images captured by shining different lasers and exciting different florescent dyes marking selective cells causing fluorescent light or the cell itself causing it to autofluorescence. The plurality of images making up a multispectral image of a cell can be used together to capture image features and numeric features to aid in classification of cell type and cell morphology. FIG. 3A illustrate using a brightfield image and a fluorescent image of four different cell images. One image in FIG. 3A is a single cell image. The other three images appear to be multiple cells that can be readily discerned by the fluorescent image and not so much by the brightfield image. The edges of the cells are easier to determine from the brightfield images.

A key benefit to using images of cells as an input is that they can simplify the classification workflow. No feature engineering or feature extraction is required. Instead, one can immediately start doing analysis on the image data of the cells that is directly output from the imaging flow cytometer. However, while using images can accelerate data exploration, it comes at a computational complexity cost. Operating on raw images consumes more computing time than operating on numeric features. This is because images maintain all available spatial data. Spatial data comes in a very high dimensional format. There is a tremendous amount of information in image data but it takes more time to process it. Another key benefit using images output from an imaging flow cytometer is that they're very easily accessible. With traditional flow cytometer event data from photo detectors or photo multiplying tubes, compensation is often required to get accurate results. With traditional flow cytometer event data, each interrogation event of a cell with a laser that is captured by photodetectors, must be preprocessed to make some sort of sense about the biological cell. In summary, images preserve all available spatial data and can be quickly collected with an AMNIS ImageStream Mk 2 imaging flow cytometer.

### Numeric Features

Referring now to FIGS. 2B and 3B, a second input into the AI analysis software are numeric features that can be extracted from the multispectral images by feature extraction algorithms. There are a number of different numeric features that can be extracted and used for AI analysis of cell images. Using feature extraction software on the images of cells, a wide range of numeric features can be extracted for use with machine learning algorithms.

FIG. 3B illustrates a table of example numeric features broken up by category. Some features that may be desirable to use by the AI software in classification include size, shape, location, texture, signal, and comparison. The size of a cell may be determined from area in the image and indicated as having a small area (left side example) or a large area (right side example) based upon a comparison with a predetermine value (e.g., 10 square nanometers, equal to and above deemed large, and less than deemed small), for example. The shape of a cell may be determined from the cell image and deemed round or lobed nuclei, for example. The location of a component (e.g., nucleus) with other parts of a cell may be determined from the cell image and deemed overlapping or separated probes, for example. The texture of the cell may be determined from the cell image and deemed diffuse or spotty. The signal level of the cell in the image can be determined and deemed dim (left side example) or bright (right side example). A comparison of the acquired cell image with a selected truth cell image can be made and a determination made whether or not they are similar images with correlation (right side example) or different with anti-correlation (left side example). Numeric features have a lower computational cost than their image counterparts, particularly when you are sorting through many images (e.g., thousands). Using numeric features as a first cut in classification can more quickly sort through a batch of multispectral images of the numerous biological cells that may be found in a sample. However numeric features obviously do not maintain all available spatial data from the source images. Additionally, if the numeric features one chooses are not comprehensive enough, the classification algorithms can still struggle classifying images of cells in a quick manner. Features generated by default, as well as custom features can be used to create a detailed and informative description of the captured image data when a user experiment is run.

In any case, numeric features are preferably a second input into the AI analysis software that can be used by the multiple AI algorithms.

### Convolutional Neural Network

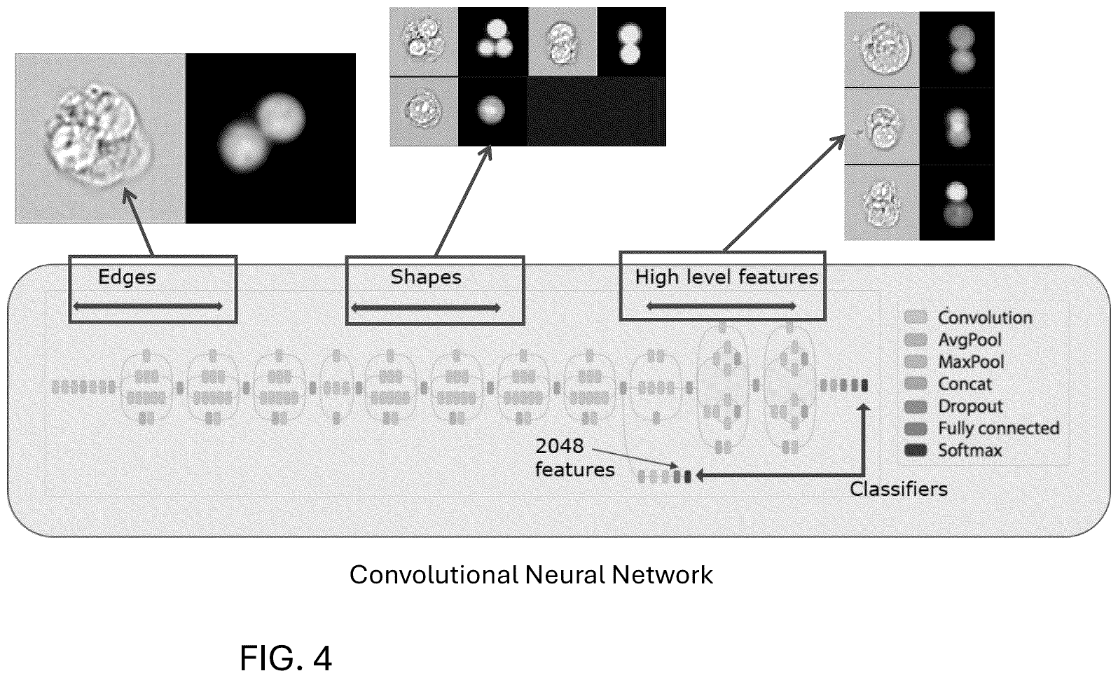

Referring now to FIG. 4, a first AI algorithm used to process each of the multispectral cellular images is a convolutional neural network (CNN) for image processing and image feature extraction. A convolutional neural network handles the high dimensionality of images by convolving over each image to learn its features and this maintains all the spatial data available. The CNN also utilizes a deep learning architecture however this makes it what's called a black box solution which means that they're not explainable. You can't step through the logic on a piece of paper to outline exactly how your CNN went from the image input to the output label.

CNNs are an industry standard in image classification. They are composed of multiple building blocks that are designed to automatically and adaptively learn spatial hierarchies of features. This enables them to handle the high dimensionality of images very well and of course as mentioned, this is what results in that black box solution. While highly effective in handling two dimensional imagery, CNNs take longer to train especially as the size of the input image grows. Thus, the CNN takes longer to train than other numeric based algorithms. The CNN in the AI Analysis software is fully optimized for biological imagery. It is pretrained to handle a diverse set of biological applications that a user is interested in with the images captured by an imaging flow cytometer.

A CNN has multiple layers (shown from left to right in FIG. 4) to process the multispectral cellular images and obtain desired image features. For example, in the earlier layers of the CNN (left side), the CNN can learn about the edges of the cell using the multispectral image. Moving deeper into layers of the CCN, other features can be discovered about the cell from the multispectral image, such as the shape of the cell. Moving into the deepest layers of the CNN, high-level image features can be determined from the multispectral image. Convolution of the image and the prior training of the parameters of the CNN extraction of image features possible. CNNs maintain all spatial data from input images and require no additional manual intervention to train. Because they use deep learning architectures, CNNs offer a specialized, deep learning method for image classification. CNNs are often considered a “black box” solution to image classification. A second algorithm in AI imaging analysis software is a random forest algorithm.

### Random Forest

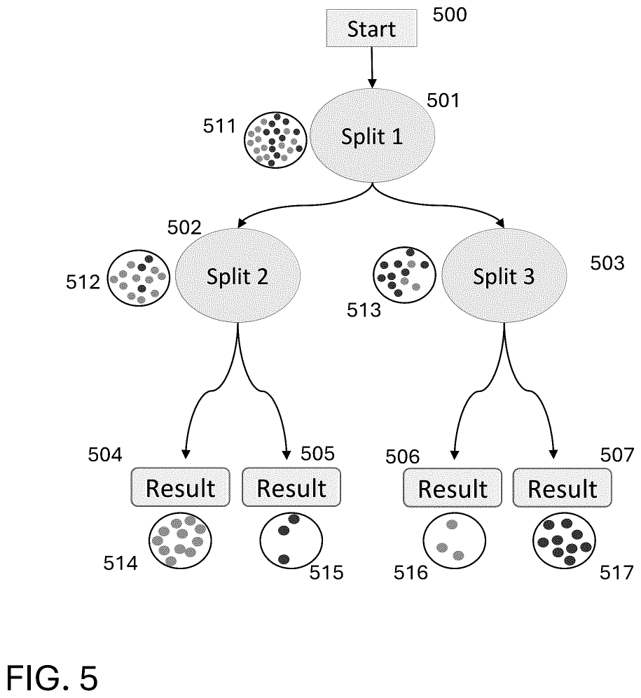

Referring now to FIG. 5, a random forests algorithm acts like one gets to choose your own adventure. The random forest algorithm start out at a starting point 500 with lots of possible options and based on the decisions that are made so one eventually narrows down to one or more final results 504-507. The random forests algorithm allows a user to gain some insight into which features have the most impact on the data in their image flow cytometry experiment. As one moves down the random forest from the start 501, the goal is to reduce heterogeneity. That is, it is desirable to increase homogeneity down the tree of the random forest. As the data is split into branches of classes, the goal is to have more of each class in each data partition.

Before a first split 501, in data partition 511 there appears to be an equal number of red and blue dots representing an equal number of different cell types. The first split 501 may be based on a numeric feature (e.g., cell area size) or an image feature (e.g., round shape) for the multispectral cell images. The first split 501 results in a data partition 512 having more blue dots than red, and a data partition 513 having more red dots than blue. At a next level, a second split 502 can be performed on the data partition 512 and a third split 503 can be performed on the data partition 513. The second split 502 on the partition 512 results in all blue dots in data partition 514 for result 504 and all red dots in partition 515 for result 505. The third split 503 on the partition 513 results in all blue dots in data partition 516 for result 506 and all red dots in partition 517 for result 507. Thus, as the algorithm moves down levels or branches and continues splitting, gradually a majority of a single class falls into each data partition 514-517.

A random forest algorithm has a couple strengths that can make them very powerful. The first is that a random forest algorithm can handle high dimensionality of numeric data very well. A random forest algorithm can also handle multiple types of features whether they be continuous or categorical. The random forest is also robust as to outliers and to unbalanced data such that a random forest results in a low bias moderate variance model.

### Modeling Pipeline

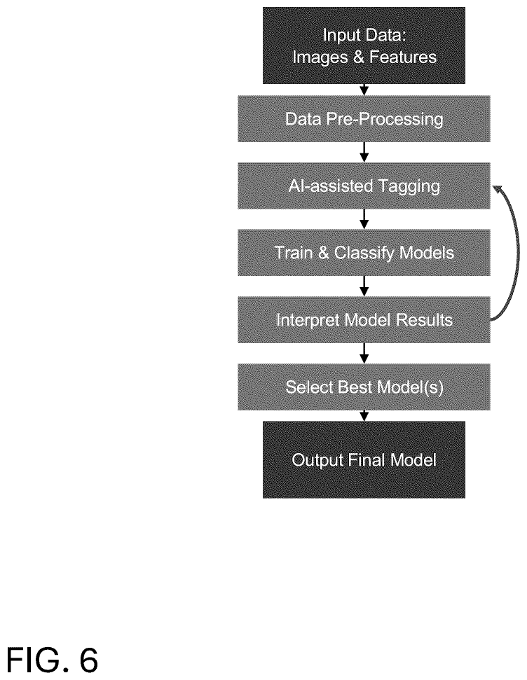

Referring to FIG. 6, the AI analysis software of the images captured by the imaging flow cytometer provides access to multiple machine learning and AI algorithms. This changes the analysis pipeline some because as you walk through the data/image analysis process, you want to evaluate which model may works best for the experiment you are trying to run. You can still iteratively tag data using AI-assisted clustering. However, you can train models using different available AI algorithms on the same input data and compare output results to determine which AI algorithm is better to use. A user can also output reports of the results and save user trained algorithms for future classification experiments.

Notice here where we want to interpret the model results on the left. It may be that you can use the output of one model to inform some information about your data. Maybe both models are struggling to classify the same classes, and you can use that information to go back and revise your tagged data in order to optimize your performance and start to get better results. Most importantly, a flexible machine learning pipeline lets you find the best model for your data. All data sets are different, and your needs are different. This flexible pipeline really allows you to adapt it to your needs.

### Example Case Study

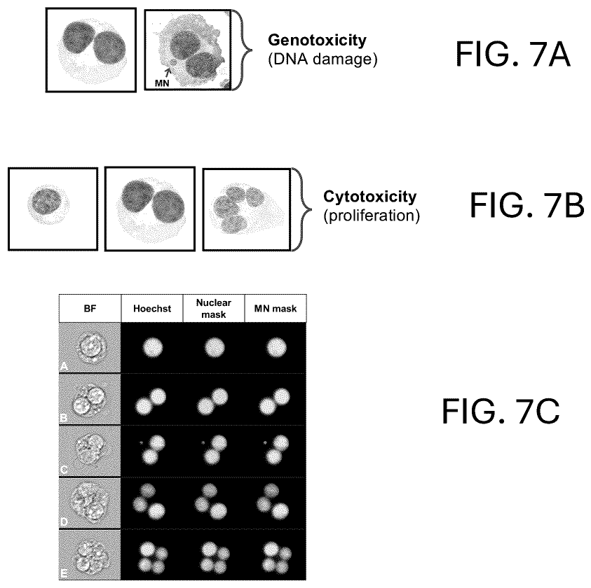

Referring now to FIGS. 7A-7C, an example of using the AI imaging analysis software is now discussed that addresses micronuclei population assignment. The in vitro micronucleus assay is an established test for evaluating genotoxicity and cytotoxicity. Performing the assay through manual microscopy is labor intensive and is very prone to inner score variability. You can use the feature extraction software first to identify populations using human engineered features in gating. However the workflow that comes from that is not very flexible. Variations in new data and identifying robust features requires expert knowledge. However, the AI imaging analysis software can help. Using AI imaging analysis software with the micronuclei images as inputs for the convolutional neural network, one can achieve greater than 95 percent accuracy in identifying micronuclei and all key events. The CNN did not require any feature calculation or gating, The CNN handles variation in the data successfully so that is it much more robust and effective for long-time analysis.

### BACKGROUND

In FIG. 7A, the micronuclei represents DNA damage. In FIG. 7B, the micronuclei represents proliferation (Cytotoxicity).) The assay is generally used for genetic toxicology, radiation bio dosimetry, and biomonitoring. It's typically scored via visual microscopy which requires skilled operators and is highly subjective. It can be scored using slide scanning microscopy which requires very high-quality slides. You can use flow cytometry which doesn't give you any imagery and also often results in false positives and you can use imaging flow cytometry. However, this requires intensive feature engineering.

### Setup

We set up our experiment via a couple of steps. First treat cells with colchicine to induce micronuclei and cytochalasin-B to block cytokinesis. Harvest, fix, and stain cells with Hoechst dye to label DNA. Run the cells and collect channel 1 (Brightfield) images and channel 7 (Fluorescent) nuclear images on an AMNIS IMAGESTREAM Mk II imaging flow cytometer.

To analyze the data, three steps were use. The first was the image files were processed with the feature extraction software to remove unwanted images. A gold standard truth series of images were created for each class that we wanted to classify in the experiment. FIG. 7C illustrates a set of truth series of images with Brightfield (BF), Hoechst, Nuclear mask and micronuclei (MN) mask. The files were processed using the AI imaging analysis software to classify each class within the model.

### Data Overview

Before we open up AMNIS AI itself and start taking a look at the data and of course what it looks like in the software here's kind of an overview of the data itself. We start we have six classes: mono, mono with micronuclei, BNC, BNC with micronuclei, multinucleated, and irregular morphology. We have 325,000 objects in the experiment. Of those objects, 31,500 have a truth label. Class balancing is handled internally by the AI imaging software, and the data is split into eighty percent training ten percent testing and ten percent validation.

The software and its algorithms analyze flow cytometry data from imaging flow cytometers. Imaging flow cytometers collect bright field, side scatter, and up to 10 colors of fluorescence simultaneously and at high throughput allowing users to collect tens of thousands of images of biological cells.

A user can use statistical image analysis software to effectively mine an image database and discover unique populations based not only on fluorescence intensity but the morphology of that fluorescence as well. IDEAS traditional analysis software used masking and feature calculation to perform image analysis. However, to accommodate increasing complexity and need for automation of image-based experiments, two new approaches to doing data analysis are provided.

The machine learning module in AMNIS AI ideas software (feature extraction software) also allows a user to create dot plots and histograms, create statistics tables, and customize the display to view the cells as single colors or any combination of overlaid images of the user needs. It also integrates seamlessly with the AMNIS AI software (AI analysis software) and houses the machine learning module and allows our users to generate publication quality reports. The machine learning module allows the user to hand tag two or more populations and then create a customized feature optimized to increase the separation of the negative and positive control samples for the user's individual experiment. It works by creating and combining the best features available in ideas using a modified linear discriminant analysis algorithm to create a super feature that is specifically tailored to the user's experimental goals.

The AI analysis software is a standalone software package that allows users to leverage the power of artificial intelligence to analyze their image data. The software will also generate a model by deep learning using convolutional neural networks to classify all user-defined populations in a sample. It in complete it includes computer aided hand tagging clustering in object map plots and creates a confusion matrix and accuracy analytics to determine how effective the model is at predicting future test samples. A comprehensive suite of image analysis software is provided including tools using artificial intelligence to simplify and strengthen the analysis of a user's image-based experiments.

### Computer Support

FIG. 1B illustrates a computer network or similar digital processing environment 10 in which the AI imaging flow cytometer system 100 can be implemented. Client computer(s)/devices 50 and server computer(s) 60 provide processing, storage, and input/output devices executing application programs and the like to implement the functionality of the feature extraction device/software 106, the AI imaging analysis device/software 107, and the database 108. Client computer(s)/devices 50 can also be linked through communications network 70 to other computing devices, including other client devices/processes 50 and server computer(s) 60. Communications network 70 can be part of a remote access network, a global network (e.g., the Internet), a worldwide collection of computers, a cloud computing environment, Local area or Wide area networks, and gateways that currently use respective protocols (TCP/IP, Bluetooth, etc.) to communicate with one another. Other electronic device/computer network architectures, such as an internet of things, and the like are suitable.

FIG. 1C is a diagram of the internal structure of a computer (e.g., client processor/device 50 or server computers 60) in the computer system of FIG. 1B. Each computer 50, 60 contains system bus 79, where a bus is a set of hardware lines used for data transfer among the components of a computer or processing system. Bus 79 is essentially a shared conduit that connects different elements of a computer system (e.g., processor, disk storage, memory, input/output ports, network ports, etc.) that enables the transfer of information between the elements. Attached to system bus 79 is I/O device interface 82 for connecting various input and output devices (e.g., keyboard, mouse, source feed or access to acquired images 111, displays, monitors, printers, speakers, etc.) to the computer 50, 60. Network interface 86 allows the computer to connect to various other devices attached to a network (e.g., network 70 of FIG. 1B). Memory 90 provides volatile storage for computer software instructions 92 and data 94 used to implement disclosed embodiments (e.g., the feature extraction device/software 106 and the AI imaging analysis device/software 107, database 108 detailed above). Disk storage 95 provides non-volatile storage for computer software instructions 92 and data 94 used to implement the disclosed embodiments. Central processor unit 84 is also attached to system bus 79 and provides for the execution of computer instructions 92 to perform the functions of the feature extraction device/software 106 and the AI imaging analysis device/software 107 illustrated in FIG. 1A.

The flow of data and processor 84 control is provided for purposes of illustration and not limitation. It is understood that processing may be in parallel, distributed across multiple processors, in different order than shown or otherwise programmed to operate in accordance with the principles of the disclosed embodiments.

In one embodiment, the processor routines 92 and data 94 are a computer program product (generally referenced 92), stored in a computer readable medium (e.g., a removable storage medium such as one or more DVD-ROM's, CD-ROM's, diskettes, tapes, etc.) that provides at least a portion of the software instructions for the system. Computer program product 92 can be installed by any suitable software installation procedure, as is well known in the art. In another embodiment, at least a portion of the software instructions may also be downloaded by communication protocols using a wired cable connection and/or wireless connection over a computer network.

The embodiments of the invention are thus described. While embodiments of the invention have been particularly described, they should not be construed as limited by such embodiments, but rather construed according to the claims that follow below.

While certain exemplary embodiments have been described and shown in the accompanying drawings, it is to be understood that such embodiments are merely illustrative of and not restrictive on the disclosed embodiments, and that the disclosed embodiments not be limited to the specific constructions and arrangements shown and described, since various other modifications may occur to those ordinarily skilled in the art.

What is claimed is:

1. A method for processing multimode images acquired by an imaging flow cytometer, the method comprising:
receiving high resolution images of a plurality of moving biological cells captured by an imaging flow cytometer from a stream of a fluid, wherein the imaging flow cytometer combines fluorescence sensitivity of standard flow cytometry with spatial resolution and quantitative morphology of digital microscopy, wherein the high resolution images acquired of each of the plurality of moving cells includes a brightfield image, a side scatter image, and a plurality of different fluorescent images respectively associated with a plurality of different spectral bands of fluorescent channels that are spatially aligned to each other;
analyzing the high resolution images of the plurality of moving biological cells to extract cellular features for each of the plurality of moving biological cells;
analyzing the high resolution images of the plurality of moving biological cells using a deep learning artificial intelligence algorithm to extract image features for each of the plurality of moving biological cells; and
analyzing the extracted cellular features and the extracted image features using a random forest algorithm to classify cell type of a plurality of cell types for each of the plurality of moving biological cells.

2. The method of claim 1 wherein:
the analyzing of the high resolution images of the plurality of moving biological cells to extract the cellular features for each of the plurality of moving biological cells is performed by a machine learning algorithm.

3. The method of claim 1 wherein:
the analyzing of the high resolution images of the plurality of moving biological cells to extract the cellular features for each of the plurality of moving biological cells is performed by the deep learning artificial intelligence algorithm.

4. The method of claim 1 wherein:
the deep learning artificial intelligence algorithm is a convolutional neural network having a plurality of artificial neurons.

5. The method of claim 3 wherein:
the deep learning artificial intelligence algorithm is a convolutional neural network having a plurality of artificial neurons.

6. The method of claim 1 wherein:
the extracted cellular features include one or more of.

7. The method of claim 1 wherein:
the extracted image features include one or more of.
